(19) United States
(12) Patent Application Publication
(10) Pub. No.: US 2016/0175610 A1
(43) Pub. Date: Jun. 23, 2016

(54) DERMAL REFLECTANCE SENSOR METHOD AND STYSTEM FORCALCULATING UV LIGHT AND VITAMIN D ABSORPTION

(71) Applicants: Troy William Livingston, Buffalo Grove, IL (US); Daniel Livingston, Buffalo Grove, IL (US)

(72) Inventors: Troy William Livingston, Buffalo Grove, IL (US); Daniel Livingston, Buffalo Grove, IL (US)

(21) Appl. No.: 14/970,456

(22) Filed: Dec. 15, 2015

Related U.S. Application Data
(60) Provisional application No. 62/093,279, filed on Dec. 17, 2014.
(60) Provisional application No. 62/096,736, filed on Dec. 24, 2014.

(51) Int. Cl. A61N 5/06; G01J 1/42

(52) U.S. Cl. CPC A61N 5/0616; G01J 1/429; A61N 2005/0627; A61N 2005/0636; A61N 2005/0654; A61N 2005/0651; A61N 2005/0661

(57) ABSTRACT

A dermal reflectance sensor method and system for providing and calculating UV light absorbed and vitamin D produced by a patient is provided. The method and system allows for real-time accurate calculating of UV light absorbed and vitamin D produced. The method and system utilizes at least five parameters for capturing and calculating data related to the specific vitamin D production of a patient.

### CROSS REFERENCE TO RELATED APPLICATIONS

The following application is based on U.S. provisional application Ser. No. 62/093,279 filed on Dec. 17, 2015 and Ser. No. 62/096,736 filed on Dec. 24, 2015 both currently co-pending, and claims the priority benefit of the '279 and the 736 U.S. applications; the entire contents of which are incorporated by reference.

### BACKGROUND OF THE INVENTION

A dermal reflectance sensor method and system for calculating UV light and vitamin D absorption in a patient is provided. The present method and system allows for real-time accurate calculating of UV light absorbed and vitamin D produced during a UV light therapy session in the present light emitting device. The present method and system utilize at least five parameters for capturing and calculating data related to the specific UV light absorbed and vitamin D produced by a patient and then altering a light therapy machine to provide optimal UV light exposure. The parameters include: 1) measuring the light source output during a specific time; 2) calculating the specific wavelength of light reflected by the skin of the patient; 3) calculating the distance the patient is from the lamp and 4) measuring the temperature of a sensing module and applying a temperature compensation factor that adjusts a calculation used; and 5) providing a safety mechanism that limits dosage to a percentage increase within a specified range, which is based upon a calculation of historical exposures and typical dosage increases, and thereby minimizing the risk of accidental operator overdosing. Finally, the present light emitting device calculates the real-time absorption of UV light and production of vitamin D, which allows an operator to control the light emitting device accordingly in real-time to optimize the proper dosage, including even the ultimate vitamin D produced in the treatment.

The modern use of sunscreens and low levels of exposure to sunlight particularly in latitudes higher than Atlanta, have proven to cause extensive deficiencies in children and adults. According to Medical News Today, 24 Aug. 2009, 70% of children in the United States are Vitamin D insufficient or deficient as defined as less than 29 nanograms of Vitamin D per milliliter of blood. The inability to accurately measure the absorbed dosage of UV light has limited the ability of the light therapy industry from creating a in system to safely and accurately expose patients to UV light for proper UV light and vitamin D therapy. This invention creates an accurate measurement of continuous real-time absorbed dosage of UV at specific and selectable wavelengths and allows the therapist to accurately dispense UV illumination with measured joules of photon energy per square meter of skin. This accurate measurement and the ability of this invention to measure reflected energy provide the data required to control the light therapy procedure and to create a medically approvable level of photon energy defined as a Maximum Erythema Dosage (M.E.D.), (the smallest dosage of UV radiation to create erythema (reddening) of the skin 24 hours after exposure). Vitamin D production by UVB exposure is then calculated by published conversion tables.

It is long established that vitamin D is primarily created through skin exposure to UVB, ultraviolet light in the spectrum that extends from 280 to 320 nanometers in wavelength. Additionally, adequate vitamin D levels are essential to the proper functioning of the human body. “Medical News Today, 24 Aug. 2009” defined vitamin D as having significant impact in preserving the genes for healthy resistance to illnesses including cancer, autoimmune, cardiovascular, and infectious diseases. Human skin sensitivity to UVB varies significantly from individual to individual and changes with each exposure to UV radiation. In addition, the accurate measurement of absorbed dosage of UVB light energy in the light therapy procedure is essential for the physician to determine the proper dosage to treat various medical conditions.

Quantifiable absorbed UVB dosage data allows the physician to adjust exposure for optimum benefit and minimum risk in the light therapy process. Prior attempts to accurately determine dosage have been limited. One method is a subjective questionnaire, the “Fitzpatrick Skin Type Chart” and uses the combined score of this test as the basis for skin sensitivity in determining maximum UV light exposure time. In addition to the Fitzpatrick questionnaire, a photon energy level meter has been utilized by some devices but this method only records UV lamp/diode output. Combination of the Fitzpatrick subjective test with light output energy does not create the data required to accurately measure an individual's skin sensitivity to burning (erythema) and the actual UV light energy absorbed by the skin. Multiple UV light exposure testing is another method of determining an individuals' M.E.D. but this procedure is time consuming, results in erythema (sunburn) and does not account for the changes in skin sensitivity with each exposure.

The present method and system measures five parameters of the light therapy process allowing for the first time accurate absorbed dosage measurements and the calculation of Vitamin D production. The first parameter is light source output measurement with typical diminishing light output continuously compensated for in total exposure time calculations. Second, a band pass filter and photometer is incorporated to measure and quantify specific wavelength of light reflected by the skin. Third, the distance of patient to light source is measured for automatic compensation of light therapy exposure time. Fourth, temperature of the sensing module assembly of two photometers, band-pass filters, distance measuring ultrasonics, and compensating electronics is monitored for input to the controlling microprocessor. Fifth, providing a safety mechanism which limits dosage to a percentage increase within a specified range, which is based upon a calculation of historical exposures and typical dosage increases, and thereby minimizing the risk of accidental operator overdosing. Each of the five measured system parameters and their relative compensation calculations are used by the system microprocessor to create a real-time patient dosage value joules per square meter. System parameters and patient position are also continuously monitored and are used to automatically interrupt the procedure if safety or dosage prescription levels are exceeded.

Patient safety, UV light toxicity, and effectivity have all been limited by subjective answers to questionnaires and physician estimation of therapy time and dosage. The present method and system will eliminate over and under exposure of light therapy and the resultant danger to the patient of ineffective or potentially harmful treatment. Accurate absorbed dosage data and individualized skin sensitivity measurement provide the data required for physicians to optimize dosage and the light therapy medical procedure.

Attempts have been made to accurately calculate UV light exposure. For example, U.S. Pat. No. 9,068,887 to Bennouri discloses a UV dosimetry system having a wearable unit and a mobile computing device. The wearable unit measures the UV irradiance intensity and wirelessly communicates with the mobile computing device. The UV dosimetry system supports multi-user control and can link one mobile computing device with multiple wearable units. The UV dosimetry system processes the measured UV irradiance intensity to calculate the UV index (UVI) and the sensor site specific UV dose. It can also calculate the total absorbed UV dose and vitamin D production by taking into account user specific factors. The UVI data measured by a plurality of UV meters such as the disclosed UV dosimetry system are crowd sourced to a remote server together with the location and time data of the measurement. The remote server excludes invalid UVI measurement and generates UVI maps showing time-varying distribution of UVI data at different locations.

Further, U.S. Pat. No. 8,793,212 to McGuire discloses a system for managing a user's exposure to the ultraviolet radiation including a user input interface, display circuitry including a screen, control circuitry including at least one processor wherein the control circuitry is configured to communicate with the display circuitry and the user input interface, data storage means for storing program instructions that, when implemented by the control circuitry, are configured to determine UV index information corresponding to a user's location, communicate with the user input interface to retrieve information for at least one user parameter, calculate at least one recommended exposure time based on the UV index information and the user parameter information, and communicate with the display circuitry to display the recommended exposure time.

However, these patents fail to provide an accurate method and system for calculating the absorption of UV light absorbed by the skin in a person receiving light therapy and instead focus upon general UV exposure from the sun while outside. A need, therefore, exists for an improved method and system of calculating UV absorption and vitamin D production and wherein an operator may adjust the therapy device based on real-time information acquired by the calculations.

### SUMMARY OF THE INVENTION

A dermal reflectance sensor method and system for calculating UV light and vitamin D absorption in a patient is provided. The present method and system allows for real-time accurate calculating of UV light absorbed and vitamin D produced during a UV light therapy session in the present light emitting device. The present method and system utilize at least five parameters for capturing and calculating data related to the specific vitamin D production of a patient. The parameters include: 1) measuring the light output during a specific time; 2) calculating the specific wavelength of light reflected by the skin of the patient; 3) calculating the distance the patient is from the light source and 4) measuring the temperature of a sensing module and applying a temperature compensation factor that adjusts the calculations used; and 5) providing a safety mechanism that limits dosage to a percentage increase within a specified range, which is based upon a calculation of historical exposures and typical dosage increases, and thereby minimizing the risk of accidental operator overdosing. Finally, the present light emitting device calculates the real-time production absorption of UV light and production of vitamin D, which allows an operator to control the fight emitting device accordingly in real-time to optimize the proper dosage.

Vitamin D is a steroid fat-soluble pro-hormone that is instrumental in the human absorption of calcium and phosphorus. Sufficient quantities of UVB tight provide adequate levels of Vitamin 02 (ergocalciferol) and 03 (cholecalciferol) the two types of Vitamin D found to be most important to the proper maintenance of the immune system. Vitamin D is created in the human body in the 3rd layer of skin by exposure to natural or artificial lighting in the UVB range of 280 to 320 nanometer (nm) wavelength. Vitamin D has been proven to boost the human immune system. The Institute of Physics, National Academy of Sciences of Ukraine, 252022 Kiev-22, Ukraine, New Method of UV Dosimetry have shown treatment with UVB light therapy is beneficial to the treatment of autoimmune diseases such as Parkinson's, cystic fibrosis, psoriasis, and cancer. These medical studies have been limited by a lack of ability to accurately measure the absorbed dosage of UVB light illumination by the test patients.

Human testing protocols are based on a subjective questionnaire called the “Fitzpatrick” test. This series of questions creates a numerical value on each of the test patient answers as to how easily they sun tan or the degree they sun burn with varying times of sun exposure and their opinion of their skin color. This subjective test is subsequently used to assign a skin type to each individual in a range from 1 to 6. The 1 through 6 number are utilized to determine the exposure time protocol for that patient's future treatment. The creation of Vitamin D by UVB radiation exposure to human skin and the varying amounts of Vitamin D produced by this exposure are different for each skin type. A problem with using the Fitzpatrick test is that it is a subjective opinion based on the patient's memory and understanding of the questions. Additionally no method exists to accurately measure the absorbed UV light energy by the patient's skin. Further complicating the task of creating a non-subjective testing protocol is each exposure to UVB light changes the patient's skin resulting in a new exposure time before sunburn occurs and the rate of absorption of UVB tight energy by skin. The present method and system addresses these issues by creating a device for use in establishing the patients initial skin type and in continually monitoring the absorbed dosage of UV light energy received by the patient during treatment. For the first time medical studies of UV light energy exposure can be accurately quantified. Treatment times and light energy output can be measured and affects of treatment can be determined to maximize beneficial medical affects. Risk of injury white testing and negative complications can be reduced and nearly eliminated by the present method and systems microprocessor (69), FIG. 19, by limiting test protocols to established safe UV exposure limits. The present method and system with the ability to accurately measure UV light energy effectiveness on an individual basis will create a safe and effective methodology for medical science to create new treatment protocols and utilize UVB light energy as an effective treatment for diseases in humans. The present method and system measure a specific and selectable wavelengths of ht produced by fluorescent tubes light emitting diodes and any and all other light sources.

A second measurement sensor measures a collimated beam of light energy reflected by the patient's skin. A microprocessor, FIG. 19, (69), incorporates the two measurements with temperature and distance inputs to create an initial skin type based on actual measured values. Testing protocols are then established by the microprocessor calculations to be used and automatically adjusted for each subsequent treatment. The present method and system allows the medical profession to meet the rigid requirements of accurate and repeatable results in testing and treatment. For this reason the present method and system will provide a valuable advancement in light therapy by enabling the creation of new protocols for the treatment of diseases in humans.

An advantage of the present dermal reflectance sensor and method is that the present method and system allows for providing a safe and accurate level of UV light absorbed and vitamin D produced by a patient.

For a more complete understanding of the above listed features and advantages of the dermal reflectance sensor method and system for calculating UV light absorbed and vitamin D produced, reference should be made to the following detailed description of the preferred embodiments and to the accompanying drawings. Further, additional features and advantages of the present method and system are described in, and will be apparent from, the detailed description of the preferred embodiments and from the drawings.

### BRIEF DESCRIPTION OF THE DRAWINGS

FIG. 1 illustrates a perspective front view of the preferred embodiment of the present light emitting device with six, four-bulb lamp/diode panels in vertical use orientation.

FIG. 2 illustrates a top view of the present light emitting device with the six lamp/diode panels in an embodiment in an open use orientation.

FIG. 3 illustrates a top view of the present light emitting device with the six lamp/diode panels in a semi-folded second orientation.

FIG. 4A illustrates the UV shield assembly in a folded configuration to shield a normal height patient's eyes from the UV radiation.

FIG. 4B illustrates the UV shield unfolded to its lower most downwardly position to shield the eyes of short or wheel chair seated patients.

FIG. 4C illustrates the UV shield folded to its upper most extended position to allow the upper most portion of UV lamp/diodes to illuminate tall patients.

FIG. 5 illustrates a vertical view of the present light therapy device with a patient in a rearward out of position location with respect to position sensor.

FIG. 6 illustrates a top view of the present light therapy device with a patient in a generally center most position with respect to tamp/diode panels.

FIG. 7 illustrates a front view of display panel showing the location of touch screen and removable memory in an embodiment.

FIG. 8 illustrates a front most view of a patient positioning indicator and emergency stop button.

FIG. 9 illustrates a front detailed view of the UV dosage sensor assembly of temperature sensor, reference light emergency sensor, reflection light energy sensor and distance sensor.

FIG. 10 illustrates a side view of the sensor assembly showing a horizontal orientation of reflected light energy sensor and 5° upward most angle orientation of reference light energy sensor with horizontal most orientation of sensor assembly.

FIG. 11 illustrates a top view of the sensor assembly showing the temperature sensor imbedded in a slot and from left to right the reference light energy sensor, the temperature sensor, the reflection light energy sensor, and distance sensor; respectively.

FIG. 12 illustrates the front most view of vertical positioning assembly and horizontal orientation of sensor assembly.

FIG. 13 illustrates the introduction page on the touch screen display.

FIG. 14 illustrates the patient entry page on touch screen display.

FIG. 15 illustrates the exposure entry in joules page on the touch screen display.

FIG. 16 illustrates the stop treatment screen on the touch screen display.

FIG. 17 illustrates the emergency stop display on the screen display.

FIG. 18 illustrates the absorbed UV illumination energy quotient delivered by the present method and system to patient.

FIG. 19 is a block diagram of the electrical inputs and outputs of the present method and systems preferred embodiment for generating UV light energy, sensing UV light energy, controlling UV light energy producing components, and displaying light energy dispensed to patient.

### DETAILED DESCRIPTION OF THE PREFERRED EMBODIMENTS

A method and system for calculating UV light and vitamin D absorption in a patient is provided. The present method and system allows for real-time accurate calculating of UV light absorption and vitamin D production during a UV light therapy session in the present light emitting device. The method utilizes at least five parameters for capturing and calculating data related to the specific vitamin D production of a patient. The parameters include: 1) measuring the lamp output during a specific time; 2) calculating the specific wavelength of light reflected by the skin of the patient; calculating the distance the patient is from the lamp; 4) measuring the temperature of a sensing module assembly of two photometers; and 5) providing a safety mechanism that limits dosage to a percentage increase within a specified range, which is based upon a calculation of historical exposures and typical dosage increases, and thereby minimizing the risk of accidental operator overdosing. Finally, the present light emitting device calculates the real-time UV light absorption and production of vitamin D and allows an operator to alter the tight emitting device accordingly in real-time to optimize the proper light therapy dosage.

Several embodiments of the present dermal reflectance sensor and light emitting device and method and system are set forth herein which relate to the monitoring of ultraviolet light energy and its absorption by human skin.

Electrical parameters of the dermal reflectance sensor are included with the physical construction of the 6, 4-lamp/diode panels and their construction for effective distribution of the light energy safely and effectively to humans. The preferred embodiment is shown but human skin varies significantly from person to person. Changes to the preferred embodiment will affect some people more or less effectively but will not alter the physics of this sensing and control system.

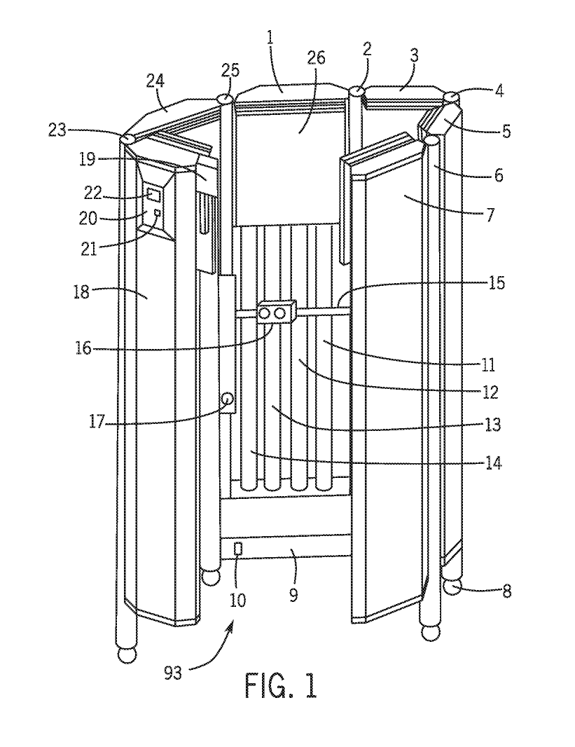

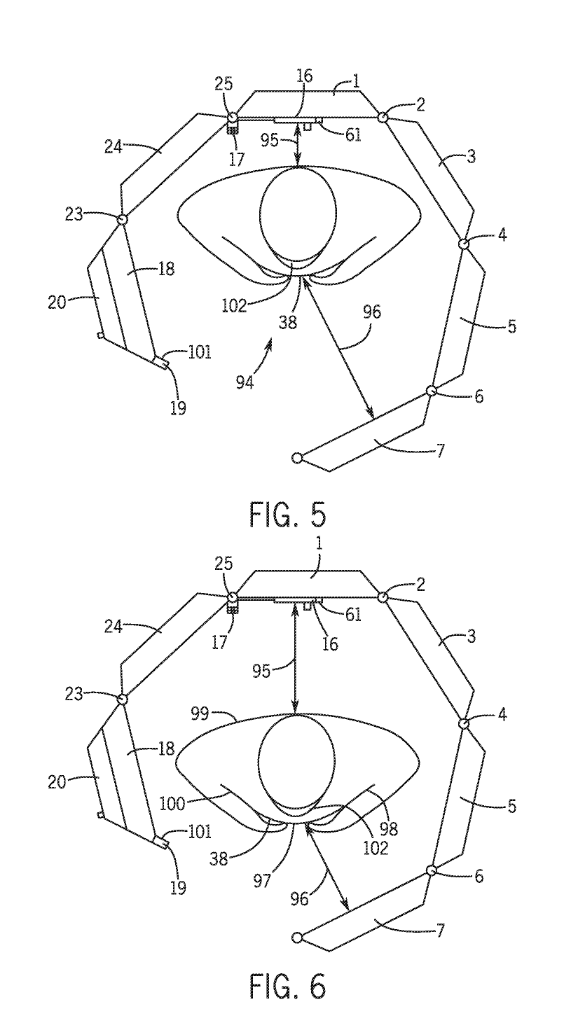

FIG. 1 illustrates the 6 lamp/diode panels (1), (3), (5), (7), (18), and (24) interconnected at their outer most vertical sides by hinge tube (2), (4), (6), (23), and (25) respectively. This physical, generally circular, open space (94) orientation allows lamp/diode panels (1), (3), (5), (7), (18), and (24) to be pivoted outwardly and inwardly creating a generally smaller or larger circular orientation (94), FIG. 5. This hinged flexibility allows the lamp/diode panel (93) to be opened or closed to accommodate thin to very large people. Sensor assembly (16) attached forward most on hinge tube (25) for providing vertical positioning adjustment via lock knob (17) for sensor assembly (16) horizontally attached top most on bracket (15). Display module (20) mounted top outside upper most on tamp/diode panel (18) receives input from sensor module (16), and touch screen (22) to measure the reference light energy from silicone detector (105), FIG. 11 a silicone detector (105), FIG. 20 or equivalent product. A band pass filter (106), FIG. 11, (106), FIG. 20 ensures only the specific and selected wavelength of light energy is passed through to silicone detector (105), FIG. 11 for measurement. The combination of reflection light energy silicone detector (48), FIG. 11 and band pass filter (50), FIG. 11 are identical to band pass filter (106), FIG. 11 and light energy silicone detector (105).

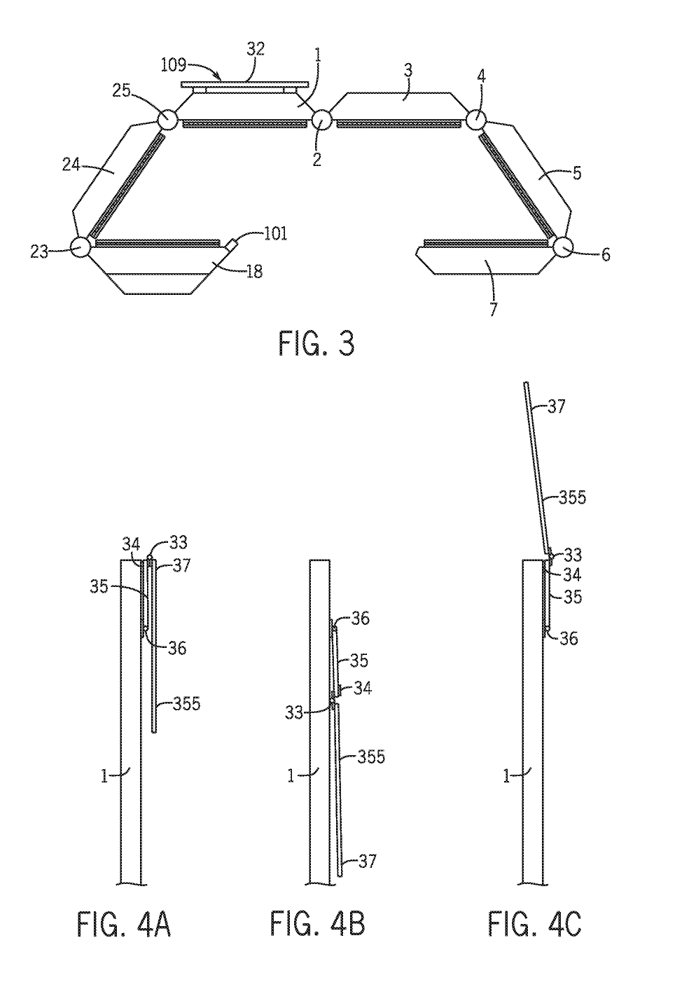

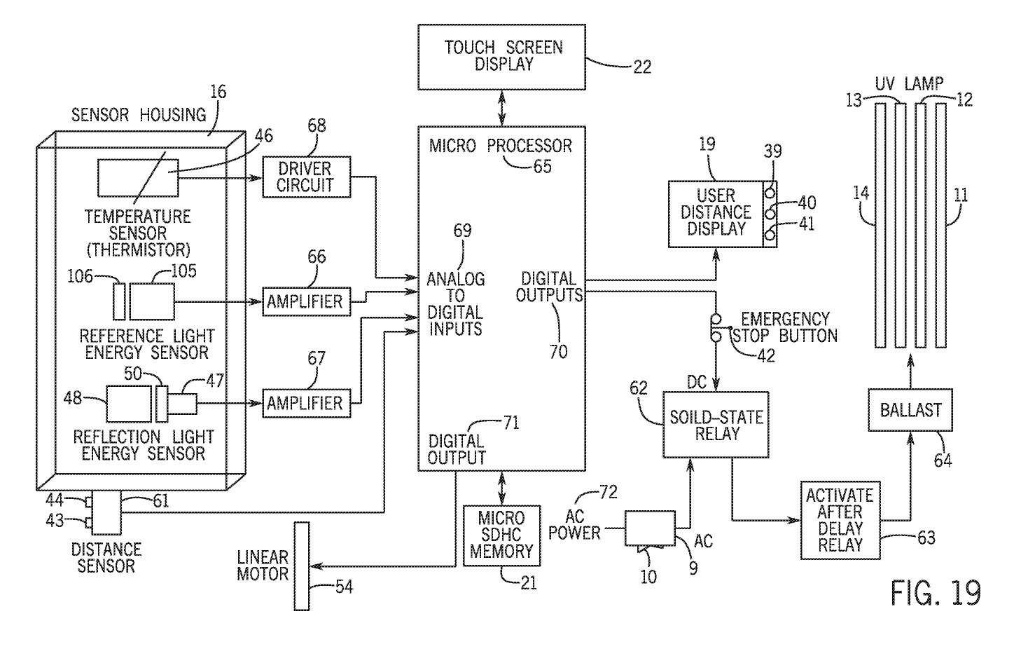

To ensure the difference in measurement, values are unaffected by manufacturing variables in sensing components for light energy at specific, selectable wavelengths. Microprocessor, (65), FIG. 19, (65), FIG. 20 also receives input from thermistor (46), FIG. 19, (46), FIG. 20 to establish temperature dependent correction factors to the input values received by light energy silicone detectors (48) and (105), FIG. 11 respectively. Distance sensor (61), FIG. 9, (61), 20 also provides input to microprocessor (65), FIG. 19 to continually monitor the patient's position in circular opening (94), FIG. 5. If the patient moves, distance measuring device (61), FIG. 9 illuminates LEDs red (39), green (40), and red (41), FIG. 8 to alert the patient they have moved too far in a forward most position with distance (95) and (96), 5 being unequal and too close to lamp/diode panel (7), FIG. 6, or in correct centered position with distance (95) and (96), FIG. 6 being equal, or have moved rearward most and in a position too close to lamp/diode panel (1) with distance (95) and (96), FIG. 5 being unequal respectively. Green LED (40), FIG. 8 will illuminate when the patient (38) returns to a center most position, in circular opening (94). UV shields (26), (27), (28), (29), (30), and (31) are shown in top most inside position on lamp/diode panels (1), (3), (5), (7), (18), and (24) respectively and will be further defined in FIG. 4. Ball castors (8) attached to the lower most end of hinge tubes (2), (4), (6), (23), and (25) are provided to support lamp/diode panels (1), (3), (5), (7), (16), and (24) during the opening and closing operation as further defined in (28), FIG. 3.

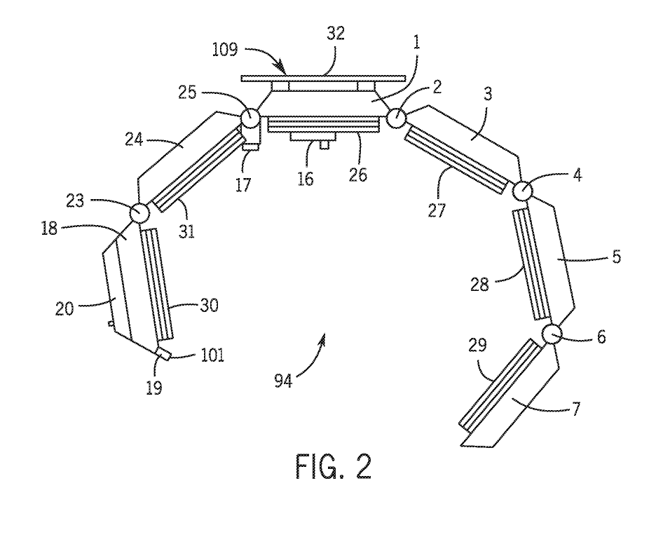

FIG. 2 is a top view of the present method and system illustrating the generally semi-circular opening (94) created by lamp/diode panels (1), (3), (5), (7), (18), and (24). The inside surface of these panels contain UV lamp/diodes (11), (12), (13), and (14) in each panel respectively. The generally circular pattern created by panels (1), (3), (5), (7), (18), and (24) therefore positions the UV lamp/diodes (11), (12), (13), and (14) respectively in equidistance surrounding patient (38).

Referring to FIGS. 5, 6 and 8, the equal distance from patient (38) of UV lamp/diodes (11), (12), (13), and (14) is essential to the present method and system ability to provide uniform radiation to patient (38) front (97), FIG. 6, left side (98), FIG. 6, back (99), FIG. 6, and right side (100), FIG. 6. Distance: display (19) inside surface (101), FIG. 8 presents three LEDs (39), (40), and (41) which alert the patient (38) of proper patient positioning (as illustrated in FIG. 5). LEDs (39), (40), and (41) respectively are positioned to be continuously monitored by patient (38), FIG. 5 to maintain proper patient positioning. Wall mounting (32) attached to the rear most surface of lamp/diode panel (1) is utilized for mounting the present method and systems lamp/diode panel (1) to any wall or supporting structure at its rearmost surface (109).

FIG. 3 illustrates a second configuration of the present light therapy device. In this second configuration, the overall required space for the device may not only be reduced, but the panels may be closer to the patient therein providing increased UV exposure and therein increased vitamin D production. Lamp/diode panels (1), (3), (5), (7), (18), and (24) are pivoted inwardly on hinge tithes (2), (4), (6), (23), and (25) respectively with lamp/diode panel (1) securely supported by wall mount (32) to any supporting surface.

FIG. 4A illustrates a side view of the UV shield (26), (27), (28), (29), (30), and (31) of the present device. The UV shield (27), (28), (29), (30), and (31) has various components (33), (34), (35), (36), and (37). UV shields (26), (27), (28), (29), (30), and (31), FIG. 2 provide eye protection for patient (38) from direct eye exposure from UV lamp/diodes (11), (12), (13), and (14), FIG. 1 in each lamp/diode panel (1), (3), (5), (7), (18), and (24), FIG. 1. Eye protection is essential to the safe operation of the present method and system. Eye protection glasses (102), FIG. 5 are required for patient (38), FIG. 5, but as an additional safety factor UV shields (26), (27), (28), (29), (30), and (31) in its center most position provide added eye protection for patient (38). This position is configured to meet the requirements of a nominal height patient (38) from 5 foot 2 inches to 6 foot. Short screen (35) is pivoted in its upward most vertical position on mounting hinge (36) and held in place by magnet (34) attracted to steel panel (1). This configuration allows long screen (37) to be suspended from its upper end most edge (102) to short screen (35) by mounting hinge (36). Long screen (37) provides eye shielding for patient (38) in this configuration for average height patients (38) in case patient (38) accidentally removes eye safety glasses (102) while being exposed to UV radiation from UV lamp/diodes (11), (12), (13), and (14) in lamp panels (1), (3), (5), (7), (18), and (24), FIG. 1 respectively.

FIG. 4B illustrates short screen (35) in its downwardly most vertical position pivoting on mounting hinge (36). Long screen (37) is hanging in its down most vertical position by pivoting on middle hinge (33) attached to short screen end (35). This configuration allows for short screen (35) and long screen (37) to extend to their lower most combined length to provide added eye protection to short patient (38) or wheel chair patients.

FIG. 4C illustrates short screen (35) pivoted to its upward most vertical position by mounting hinge (36). Long screen (37) is pivoted to its upward most vertical position by middle hinge (33). This configuration removes UV screens (26), (27), (28), (29), (30), and (31) from blocking upper end most UV tamp/diode radiation for tall patient (38).

FIG. 5 illustrates a top view of patient (38) man off-center rear most configuration with respect to circular opening (94), UV lamp/diodes (11), (12), (13), and (14) unite output energy received by patient (38) is dependent on equal spacing of lamp/diode panels (1), (3), (5), (7), (18), and (24) to front (97) left side (98), back (99), and right side (100) of patient (38). The distance display (19) illustrated in FIG. 8 has LED lamps/diodes indicating that the patient (38) is too far from (39); the correct distance to (40); or too close (41) to one of the panels of the light therapy device. If the patient (38) is too far or too close to a panel, the LED lamp/diodes indicate the same and the patient (38) therein may adjust his/her position so as to be properly located within the device.

FIG. 6 illustrates patient (38) in proper center most position within circular opening (94) for receiving uniform UV irradiation from light source (11), (12), (13), and (14) in lamp/diode panels (1), (3), (5), (7), (18), and (24) respectively. Distance sensor assembly (16), FIG. 9 continuously measures the distance (95) and provides data input to microprocessor (65), FIG. 19. Microprocessor (65), FIG. 19 provides output to LED (39), FIG. 8, (40), FIG. 8, or (41), FIG. 8 dependent on proper patient (38) positioning in circular opening (94).

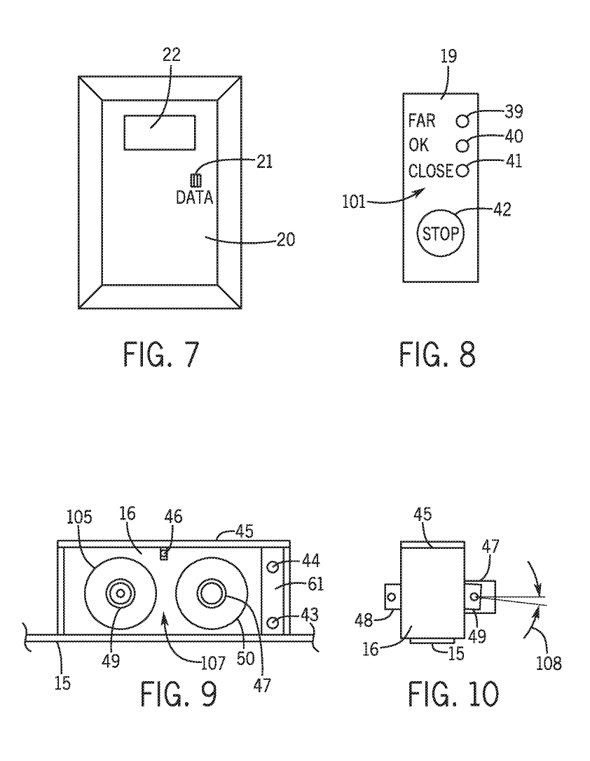

FIG. 7 is a front view illustration of the display module (20) mounted on the top outer most surface of panel (18). Touch screen display (22) is located on top most vertical surface of display module (20) for the light therapy operator to input patient data and receive therapy output data as defined in FIGS. (13), (14), (15), (16), (17), and (18) below. Micro SDHD memory (21) located on the front most surface of display panel (20) is utilized to provide transportable data storage of the present method and system's patient (38) identification and therapy protocols.

FIG. 8 is a front most surface illustration of distance display (19). Proper center most positioning of patient (38) in circular opening (94) is essential to effective UV light irradiation therapy. Top most LED “far” illuminates when distance sensor assembly detects patient (38) has moved too far in a rearward direction with respect to a center most position in circular opening (94), LED far (39) illuminates to alert patient (38) to move in a forwardly motion to a center most position within the circular opening (94). Proper patient (38) center most positioning in the circular opening (94) will cause LED “ok” (40) to illuminate and indicate to patient (8)proper therapy treatment positioning. Patient movement in a forwardly motion wilt result in LED far (41) illumination indicating to patient (38) to move in a rearward motion to return to the center most position within circular opening (94). At this position, LED ok (40) will re-illuminate indicating to patient (38) the return to proper therapeutic position. Stop button (42) is located on lower most front surface of distance display (19) for immediate easy access by patient (38) in the event patient (38) wants to stop the light therapy procedure. Pressing stop button (42) turns off UV lamp/diodes (11), (12); (13), and (14) in panels (1), (3), (5), (7), (18), and (24) respectively, and signals microprocessors (65), FIG. 19 to display exposure stopped page (81), FIG. 17. The tight therapy protocol will only resume upon the operators pressing touch screen (81), FIG. 17, continue button (82), FIG. 17.

FIG. 9 is a front most surface illustration of sensor assembly (16). Proper positioning of patient (38) within circular opening (94) is essential to uniform irradiation of patient (38) by UV tamp/diodes (11), (12), (13), and (14) irradiance from lamp/diode panels (1), (3), (5), (7), (18), and (24). Distance sensor (61), FIG. 9, (61), FIG. 20 utilizes infrared input and output LEDs (44) and (43) respectively to determine the spatial distance from the sensor (61) to a solid object, of the present method and system to the patient (38); proper positioning is essential to effective safe therapeutic UV lamp/diode (11), (12), (13), and (14) treatment.

UV light therapy effectiveness and safety is dependent on accurate measurement of the joules of UV illumination absorbed by patient (38). Initial measurement of patient (38) skin type is necessary to assign a skin type index from 1 to 4 to patient (38). This index number from 1 to 4 determines the therapeutic exposure protocol to be appropriate to each patient (38). Each protocol from 1 to 4 sets the time of exposure limits to prevent patient (38) from being overexposed to UV illumination and burned or under-treated and obtaining no medical benefit. Sensor assembly (16) contains two identical UV sensors, reference UV tight energy silicone detector (105) and reflected UV light energy detector (108). Both silicone detectors (105) and (48) receive their UV light through band pass filter (106) and band pass filter (50) respectively. UV lamp/diode energy entering band pass titter (50) is restricted to only the UV light energy passing through collimator (47). The purpose of this collimation will be explained later in this section. To measure absorbed dosage of UV light energy by patient (38), the total exposure UV light energy received by patient (38) must be accurately measured on a continuous basis. UV light energy reflected from patient (38) skin is also required to be continuously measured.

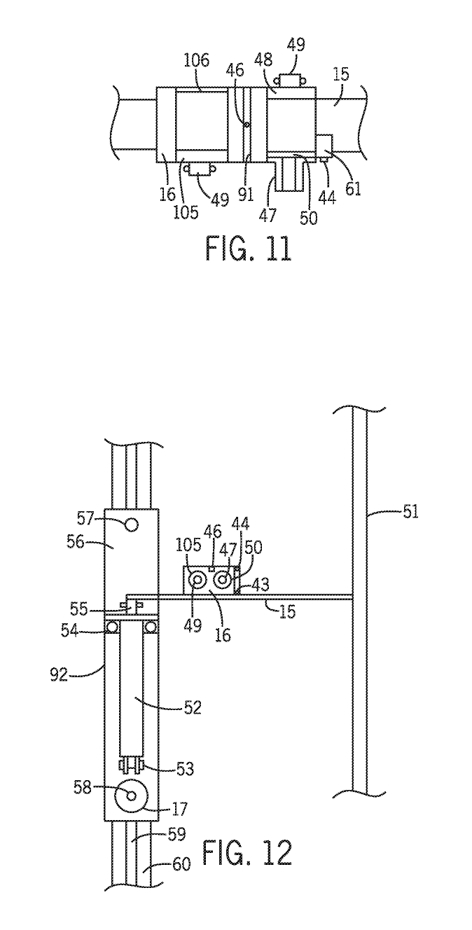

The difference in these two measurements can then be determined by microprocessor (65). Absorbed dosage of UV light energy by patient (38) is equal to reference UV light energy value minus reflected UV light energy value. Patient absorbed UV light energy=reference UV light energy reflected UV light energy. The instantaneous UV light energy absorbed by patient can then be measured over time of exposure to result in the determination of total UV light energy absorbed by patient (38) and is reported as millijoules per square centimeter of patient (38) skin. Proper positioning by patient (38) as indicated by distance display (t9) is essential to accurately measuring the UV light energy level received by patient (38), too close illuminating LED (41) close, or too far illuminating LED (38) far will result in UV light energy over or under exposure by UV light (11), (12), (13), and (14) in panels (1), (3), (5), (7), (18), and (24) respectively to patient (38). Calculations by microprocessor, (65), FIG. 19 of time required to meet skin type joules/cm² UV exposure limits will also be negatively affected by patient (38) being out of proper center position in opening (94), FIG. 6; Collimator (47) is utilized to limit reflected UV light energy reflected back to silicone detector (48) from a 6 cm diameter spot on patient (38) skin. This limitation inhibits scattered UV light energy generated by UV light (11), (12), (13), and (14) in lamp/diode panels (1), (3), {5), (7), (18), and (24) from being mixed with reflected UV light energy received through collimator (47) from the 6 cm diameter spot target on patient {38) skin. Silicone detectors (105) and (48) must be maintained at the same temperature and the actual temperature of these devices must be measured and transmitted to microprocessor (65), FIG. 19. This measurement enables microprocessor (65), FIG. 19 to create a temperature compensation factor to the output values from silicone detectors (105) and (48) for accurate measurement of UV light energy reporting by silicone detectors (47) and (105). Thermistor (46) imbedded in slot (91) provides temperature measurement values of aluminum block (107), which holds silicone detectors (48) and (105) in thermal insulation (45), FIG. 10. This insulated package design maintains silicone detectors (48) and (105) at the same temperature and insulation (45) reduces the rate of temperature change of aluminum block (107) when exposed to changes in outside temperature.

FIG. 10 illustrates a horizontal view of sensor (16) showing upwardly forwardly angle of reference silicone detector (105), connector (49) and band pass filter (106), FIG. 11. This 5° upwardly forwardly angle is required to eliminate the scattered UV light energy reflected from UV lamp/diode (12) outside most surface from interfering with silicone detector (105) UV lamp/diode (12) UV light energy output.

FIG. 11 is a top view of sensor assembly (16) showing the horizontal relationships of reference UV light energy silicone detector (105) and band pass filter (106), also reflectance UV light energy silicone detector (48), band pass filter (50), and collimator (47). Thermistor (46) is shown in center most position of block (107) for equal measurement of the temperature of silicone detectors (105) and (48). Distance detector attached further right most on block (107) is also shown enclosed in insulation (45) for maintenance of uniform temperature of all components of sensor assembly (16).

FIG. 12 is a front view of vertical positioning assembly (92). Wheelchair, short, normal, and tall patients (38), FIG. 6 must all be accommodated for the present method and system to produce effective light therapy treatment to patient (38), FIG. 6. Vertical adjustment of mounting plate (56) is provided by turning lock knob (17) causing lock shaft (58) to allow mounting plate (56) to move in an upwardly or downwardly motion. This adjustment motion allows the light therapy operator or patient to adjust sensor assembly (16) in an upwardly or downwardly motion. Sensor assembly (16) is rigidly center most mounted to sensor bracket (15), which is rigidly end most mounted to linear motor piston (55). Linear motor piston (55) is controllably moved upwardly or downwardly in a vertical motion on mounting plate (56) by linear motor (52) mounted end most rigidly on actuator mounting pin (53).

Linear motor (52) extends linear motor piston (55) 4″ in an upwardly or downwardly motion to enable sensor bracket (15) to move center most mounted sensor assembly (16) 4″ in an upwardly or downwardly motion with respect to mounting plate (52). This combination of vertical upwardly or downwardly motion of mounting plate (56) and linear motor (52) enables the light therapy operator or patient to adjust sensor assembly to meet the vertical positioning requirements of sensor assembly for varying heights of patients (38). The movement of the sensor assembly (16) therein provides for a more accurate reading of the reflected UV light from the patient (46) and therein allows for the increase or decrease of the amount of UV exposure to the patient to, for example, optimize vitamin D production.

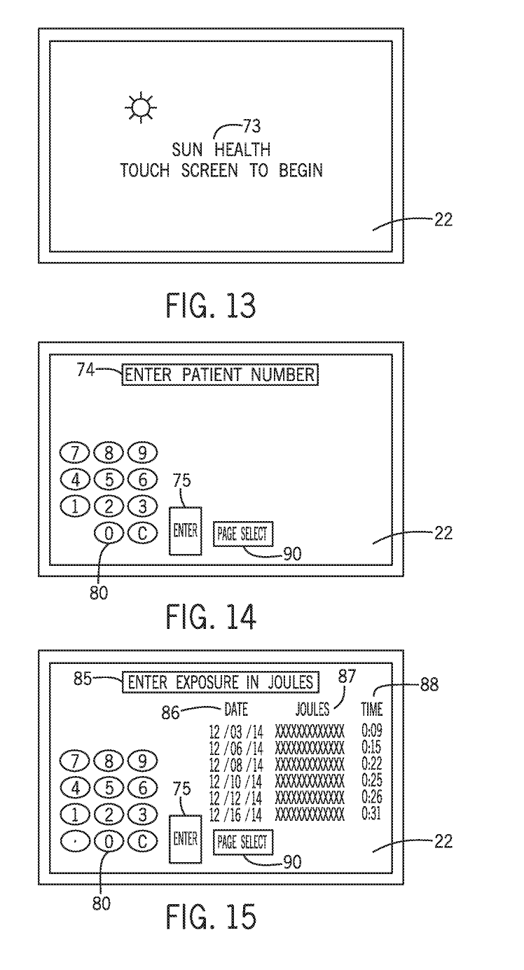

FIG. 13 is a view of touch screen (22) mounted center top most on the outer most front surface of display module (20). FIG. 13 is the introduction screen instructing the light therapy operator or patient to touch screen begin the light therapy treatment protocol.

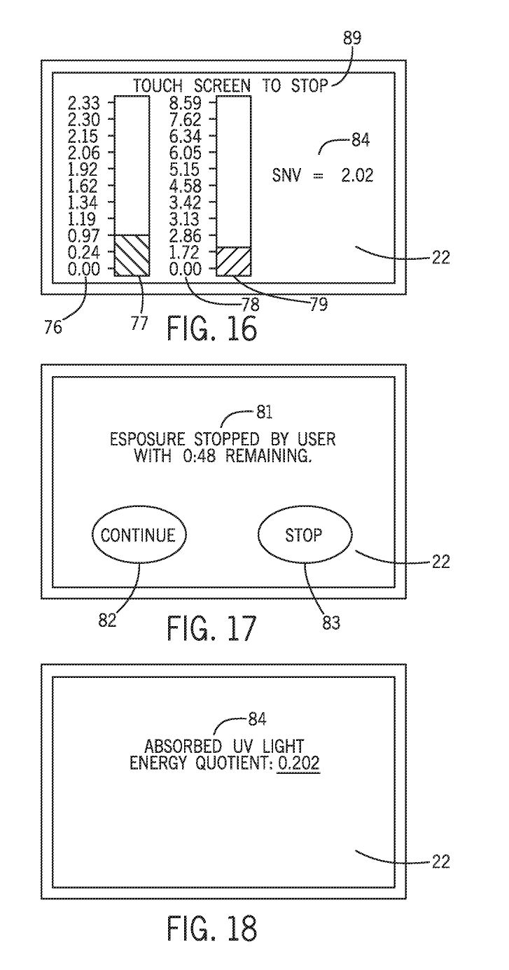

FIG. 14 is a view of touch screen (22) instructing the operator to enter patient (38) ID number on touch pad (80) and then to touch enter (75) or page select (90) to scroll through touch screens initializing message (73), FIG. 13, patient ID (74), FIG. 14, exposure (85), FIG. 1.5, stop treatment (89), FIG. 16, emergency stop (81), FIG. 17 or energy quotient (84), FIG. 18.

FIG. 15 is a view of the light energy entry screen for the light therapy operator or patient to enter the light therapy protocol as directed by the skin type determination by sensor (16). Date (86), joules of light energy (87) and exposure time (88) will be displayed by microprocessor (65) according to the selected skin type protocol.

FIG. 16 is a view of the stop treatment screen. This screen allows the light therapy operator or patient to monitor the treatment time and stop the treatment if desired. Microprocessor calculated patient absorbed dosage numerical value (84) is displayed as reference for future treatment evaluation.

FIG. 17 is a view of the screen alerting the light therapy operator that patient (38) has pressed emergency stop button (42), FIG. 8 and the time remaining in the treatment protocol. Continue button (82) can be touched to cause the therapy protocol to resume, or stop button (83) can be touched to terminate the therapy session.

FIG. 18 is a view of the last screen in the therapy treatment session with patient (38) absorbed UV light energy quotient displayed. FIG. 19 is a block diagram of electrical inputs and outputs of the present method and systems components and sensors. Microprocessor (65) receives input from distance sensor (61) through digital inputs (69) defining the 7 to 15 cm range of close: 7 to 9 cm, ok: 10 to 13 cm, or far: 14 or greater cm from sensor (16) to patient (38). Based on this input from distance sensor (61), microprocessor (65) provides output to user display (19), close (41), ok (40), or far (39) LEDs respectively. Sensor assembly (16) provides input to microprocessor (65) through digital inputs (69) for thermistor temperature (46), reference light energy value (105), and reflected light energy value (48). These inputs are then used by microprocessor (65) to receive light (13) energy output value, patient (38) reflected light energy value (48), and block (107) temperature to calculate patient (38) UV absorbed dosage.

Linear motor (54) is utilized to provide vertical most upwardly and downwardly movement for sensor (16) to measure patient (38) skin reflected light energy at two or more vertically most upwardly and downwardly scanning positions. Reflected light energy received by reflection light energy silicone detector (48) from two or more vertical most upwardly and downwardly spots on patient (38) provides verification of accuracy of the light energy reflected by patient (38). This multiple reading is analyzed by microprocessor (65) for acceptable deviation in reflected light energy value before microprocessor (65) assigns a skin type for patient (38). Microprocessor (65) outputs display information to touch screen (22) based on calculated values and inputs as noted above 110V AC power is provided by external source (72) to power input box (9) and switched on and off by power switch (10).

Mounted on outer most surface of power input box (9), solid-state relay (62) is maintained in a normally electrical current conducting mode to allow uninterrupted electrical power to microprocessor (65) for control of light source (11), (12), (13), and (14) in panels (1), (3), (5), (7); (18), and (24) respectively. Depression of emergency button (42) opens solid-state relay (62) and causes electrical power to be turned off to ballast (64) powering lamps/diodes (11), (12), (13), and (14) in panels (1), (3), (5), (7), (18), and (24) respectively. Secondary safety, activated after delay relay (63) is in series with electrical current input to ballast (64) and is set to 10 seconds or a similar delay time longer than the longest time previously selected as defined above therapy treatment protocol. If microprocessor (65) fails to function as programmed or an improper operator input to screen (85), FIG. 15 could result in patient (38) exposure time exceeding approved protocols activate after delay relay will automatically turn off electrical current to ballast (64) turning off lamps/diodes (11), (12), (13), and (14) in panels (1), (3), (5), (7), (18), and (24), FIG. 1 respectively and preventing injury to patient (38).

For all light and distance sensors, over-sampling and averaging are used to increase resolution and improve noise immunity.

Light sensor temperature compensation:

The outputs of the sensors vary depending upon their temperature. In order to get a consistent voltage output proportional to the light level, the output voltage must be compensated for this temperature effect. Temperature compensation is provided using a polynomial of the form:

V_{tc}=(Vₛ+Z₀+(Z₁*T)+(Z₂*T²))*(1+(L₁*(T−77)))

where
V_{tc} is the temperature compensated output voltage
Vₛ is voltage measured from the sensor
T is the temperature as measured by the temperature sensor
Z₀ is the constant coefficient for sensor with no UV light input
Z₁ is the linear coefficient for the sensor with no UV light input
Z₂ is the second order coefficient for the sensor with no UV light input
L₁ is the linear coefficient for the sensor with the UV light turned on.

*Note that the above polynomial is a combination of two polynomials. The second half compensates for a change in the temperature affect between when the UV light is on and off.

The constants are empirically derived for each sensor. Following are an example of values derived for one of the sensors:
Z₀ 1.659
Z₁ −3.42E-02
Z₂ 1.96E-04
L₁ −4.67E-02

To improve accuracy, both sensors are embedded in the same metal block along with a temperature sensor. This helps keep all three components close to the same temperature. In addition, the part of the metal block that does not need to be visible to the light source is wrapped in insulation. The insulation reduces the rate of change in temperature caused by external sources such as the UV lights and room air handlers.

Distance compensation for body sensor.

For the body sensor, the temperature compensated value is also compensated for distance by applying a linear equation to the compensated value calculated above. This takes the form of:

V_{dc}=V_{tc}*(1−D₁*D₁₄)

Where:
V_{dc} is the distance compensated value
V_{tc} is the temperature compensated output voltage calculated above
D₁₄ is the distance difference from 14 centimeters
D₁ is the linear coefficient

The linear coefficient has been derived empirically. An actual value derived for one of the sensors is:
D₁ 0.0374

### Skin Type Calculations:

Different skin types absorb UV light differently and the protocols used by the industry vary dependent on the skin type. A common term used in the industry is the Fitzpatrick number although other definitions are in use. Upon first use, the present system calculates a “Fitzpatrick” number and type using the following methodology:

Calculate the Body Ratio

B_{R}=V_{b}/V_{ref}

where
B_{R} is the body ratio
V_{b} is the temperature and distance compensated voltage from the sensor measuring the light reflected by the patient's body
V_{ref} is the temperature compensated voltage from the sensor measuring the light output of the bulbs

Calculate the Fitzpatrick number. This is accomplished using a linear equation that takes the form:

Fₙ=F₀+(F₁*B_{R})

where
Fₙ is the Fitzpatrick number
B_{R} is the body ratio calculated above
F₀ is the constant coefficient
F₁ is the linear coefficient

The constants have been empirically derived. An example from one of the actual systems is:
F₀ 46.4
F₁ −44.0

Associate the Fitzpatrick number to a skin type using the following groupings:
1 0-7
2 8-17
3 18-25
4 26-1
5 >31

### Joule Conversion.

Because the output of the sensor is a voltage while the exposure is tracked in Joules, a conversion factor is needed. This takes the form of:

Eᵣ=V_{tc}*C_{f}

Where
Eᵣ is the current exposure rate in milli-joules/second
V_{tc} is the temperature compensated output voltage calculated above
C_{f} is the conversion factor, for example 3.311

This conversion factor is applied to every reading of the sensor which happens several times a second. Several factors affect the conversion factor. Therefore, it is set for each individual unit.

### Exposure Summation.

During patient exposure, the system maintains a running sum of the exposure. At the start of an exposure, the exposure sum is set to 0. Then every time the sensor is read, the sensor reading is multiplied by the elapsed time since the last reading to create an exposure for the current period.

E_{c}=Eᵣ*Tₑ

where
E_{c} is the current exposure
Eᵣ is the current exposure rate in milli-Joules/second as calculated above
Tₑ is the elapsed time since the last exposure in seconds.

This current exposure is added to the exposure sum to create a total exposure for the session.

Exp=Σ(E_{c})

where
Exp is the total exposure received so far in milli-Joules
E_{c} is the current exposure
(Σ is the standard accepted symbol for summation)

Reading of the sensor takes place several times a second to provide the accuracy and control required.

Although embodiments of the present invention are shown and described therein, it should be understood that various changes and modifications to the presently preferred embodiments will be apparent to those skilled in the art. Such changes and modifications may be made without departing from the spirit and scope of the present invention and without diminishing its attendant advantages. It is, therefore, intended that such changes and modifications be covered by the appended claims.

I claim:

1) A dermal reflectance sensor method and system for providing and calculating UV light absorbed and vitamin D produced during light therapy, the system and method comprising:
providing a light therapy machine having a UV lamp/diode for providing UV light wherein the light therapy machine has a housing having a top, a bottom and a generally hollow interior wherein a patient is capable of being located within the generally hollow interior and wherein the patient has skin which may receive a light therapy treatment;
providing a first sensor within the generally hollow interior of the light therapy machine wherein the first sensor measures UV light output from the UV lamp/diode;
providing a second sensor within the generally hollow interior of the light therapy machine wherein the second sensor measures the distance from the UV lamp/diode of the light therapy machine to the patient;
providing a third sensor within the generally hollow interior of the light therapy machine wherein the third sensor measures reflected UV light from the skin of the patient;
providing a computer wherein the computer in electrical communication with the light therapy machine wherein the computer calculates vitamin D production in the patient by:
first determining n output value of UV light in terms of millijoules and time;
secondly providing a band pass filter and photometer to measure the specific wavelength of the UV light reflected off of the skin of the patient;
thirdly, measuring the distance of the patient to the UV lamp/diode;
fourthly, measuring the temperature of a sensing module and applying a temperature compensation factor that adjusts the calculation used; and
fifth, providing a safety mechanism that controls the dosage based upon a calculation of historical exposures and limits dosage to a percentage within a particular range; and
wherein an operator controls the light therapy machine in real-time to optimize a proper dosage of UV exposure to the patient.

2) The dermal reflectance sensor method and system for providing and calculating UV light absorbed and vitamin produced during light therapy of claim 1 wherein the calculating of the UV light absorbed and vitamin D produced by the computer is in real-time.

3) The dermal reflectance sensor method and system for providing and calculating UV light absorbed and vitamin D produced during light therapy of claim 1, further comprising the step of:
providing a plurality of panels which are secured at a hinge and which rotate independently with respect to each other and wherein the plurality of panels forms an enclosure of the light emitting device; and
providing an opaque UV eye shield located and secured to a front of one of the plurality of panels wherein the opaque UV eye shield has a first unit, a second unit and a hinge wherein the first unit rotates with respect to the second unit at the hinge.

4) The dermal reflectance sensor method and system for providing and calculating UV light absorbed and vitamin D produced during light therapy of claim 3 further comprising the step of:
providing a magnet secured to the front of the first unit of the opaque UV eye shield wherein the magnet may correspondingly be secured to a metallic portion of one of the plurality of panels.

5) The dermal reflectance sensor method and system for providing and calculating UV light absorbed and vitamin produced during light therapy of claim 3 wherein the opaque UV eye shield moves from a first orientation wherein the first unit and second unit are approximately one hundred and eighty degrees with respect to each other to a second orientation wherein the first unit is located between the second unit and one of the plurality of panels and wherein the first unit is therein concealed by the second unit.

6) The dermal reflectance sensor method and system for providing and calculating UV light absorbed and vitamin D produced during light therapy of claim 1 wherein the increase or the decrease of the UV light emitted from the UV lamp/diode is automatically adjusted up or down depending on if the vitamin D production is greater or lower than a preprogrammed amount.

7) The dermal reflectance sensor method and system for providing and calculating UV light absorbed and vitamin D produced during light therapy of claim 1, further comprising the steps of:
providing a motor to operate the first sensor and the second sensor wherein the motor allows the first sensor and the second sensor to move along a rail located within the interior of the housing;
wherein the movement of the first sensor and second sensor measure the skin reflected UVB light energy at various locations on the body of the patient; and
wherein the amount of UV light exposed to the patient is increased or decreased based on the information collected by the first sensor and second sensor.

8) The dermal reflectance sensor method and system for providing and calculating UV light absorbed and vitamin D produced during light therapy of claim 1, further comprising:
a silicone detector, connector and band pass filter wherein the silicone detector is angled upward between four and six degrees so as to eliminate scattered light energy reflected from the light source off of the housing.
